(19) United States
(12) Patent Application Publication
(10) Pub. No.: US 2022/0235394 A1
(43) Pub. Date: Jul. 28, 2022

(54) REVERSE TRANSCRIPTION POLYMERASE CHAIN REACTION DIAGNOSTIC TESTING DEVICE

(71) Applicants: Sandra Janeth Perdomo Lara, Bogota (CO); Justo Leonardo Calderon Mendoza, Bogota (CO); John Mario Cepeda, Bogota (CO); Alfredo Ernesto Hoyos Ariza, Miami, FL (US); Luis Eduardo Belalcazar, Bogota (CO)

(72) Inventors: Sandra Janeth Perdomo Lara, Bogota (CO); Justo Leonardo Calderon Mendoza, Bogota (CO); John Mario Cepeda, Bogota (CO); Alfredo Ernesto Hoyos Ariza, Miami, FL (US); Luis Eduardo Belalcazar, Bogota (CO)

(21) Appl. No.: 17/331,748

(22) Filed: May 27, 2021

Related U.S. Application Data
(60) Provisional application No. 63/141,221, filed on Jan. 25, 2021.

(51) Int. Cl. C12Q 1/686; B01L 7/00

(52) U.S. Cl. CPC C12Q 1/686; B01L 2300/1877; B01L 7/52

(57) ABSTRACT

A diagnostic testing device for detecting sequences of genetic material and other targeted molecules by collecting at least one sample, purifying and enriching the at least one sample for reverse transcription polymerase chain reaction, performing reverse transcription polymerase chain reaction, and indicating the presence or absence of an at least one targeted molecule or sequence of genetic material. The device is comprised of a housing which is further comprised of an exterior component, an interior component, and a testing assembly disposed therein. The device is mobile, thereby permitting convenient diagnostic use and effective storage and concealment when not in use, and laboratory-independent, thereby dismissing any need for laboratory technician intervention and input.

### CLAIM OF PRIORITY

The present application claims priority under 35 U.S.C. Section 119 to a currently pending, U.S. Provisional application having Ser. No. 63/141,221 and filed on Jan. 25, 2021 which is incorporated by reference herein in its entirety.

### BACKGROUND OF THE INVENTION

### Field of the Invention

The present invention relates to a device configured to collect, purify, and enrich at least one sample and to amplify sequences of nucleic acids via reverse transcription polymerase chain reaction (RT-PCR) in order to detect and diagnose a variety of infectious and non-infectious conditions. Further, the present invention relates to a device that is mobile, thereby permitting convenient diagnostic use and effective storage and concealment when not in use, and laboratory-independent, thereby dismissing any need for a laboratory setting or laboratory technician intervention and input.

### Description of the Related Art

Diagnostic testing is ubiquitous in the discipline of medicine and is commonly used to confirm or deny the presence of a variety of infectious and non-infectious conditions in a myriad of patients. Diagnostic testing can be invasive, wherein a patient's body must be physically intruded upon to collect an appropriate sample, or non-invasive, wherein no such intrusion is necessary. Before any diagnostic testing can occur, however, an appropriate sample or multiple samples must first be collected and prepared in a manner that ensures the efficacy of the diagnostic testing. One such method to prepare an appropriate sample for diagnostic testing is through use of a technique known as polymerase chain reaction.

Polymerase chain reaction (PCR) is a technique used to amplify, or make a large number of copies of, sequences of genetic material, allowing scientists and other individuals to take a small sample of nucleic acids and make millions to billions of copies of them for diagnostic testing and other purposes. The majority of methods currently available to perform PCR utilize thermal cycling, wherein the appropriate reactants are exposed to repeated cycles of heating and cooling in order to allow different temperature-dependent reactions to occur. PCR traditionally comprises three steps: denaturation, annealing, and extension/elongation. In the first stage, denaturation, a sample of nucleic acids is denatured, wherein each deoxyribonucleic acid (DNA) molecule's double-stranded helical structure is unwound by way of breaking the hydrogen bonds between complementary base pairs, which ultimately yields two single-stranded DNA molecules. In the second stage, annealing, the single-stranded genetic material is annealed, wherein certain DNA molecules known as primers bind to specific regions of the single-stranded DNA molecules. In the third stage, extension, an enzyme that polymerizes new DNA strands extends the primers along the template strands in the 5′-3′ direction, creating a newly synthesized DNA strand for each template strand. These three steps, known collectively as a “cycle of amplification,” are usually performed a great number of times to produce the millions to billions of copies of genetic material necessary for a variety of applications, such as diagnostic testing.

In this classic form of PCR, however, the template strand used in the annealing step is a single-stranded DNA molecule. One limitation of using DNA as a template strand for PCR is an inherent inability to detect ribonucleic acid (RNA) viruses and other RNA-related conditions by way of diagnostic testing. Therefore, one variant of PCR, reverse transcription polymerase chain reaction (RT-PCR), differs from it in that the latter uses RNA as a template instead of DNA. RT-PCR advantageously utilizes an additional step to compensate for the lack of ability to test for RNA-related condition via PCR. Specifically, in RT-PCR, any RNA present in a sample undergoes reverse transcription to complementary DNA (cDNA), a form of DNA, as cDNA is unaffected by RNase degradation (and is thus more chemically stable than RNA). Once this step has occurred, the resulting cDNA is sufficiently stable to withstand the heat produced during amplification without degrading the genetic material contained therein.

As the process of PCR was invented relatively recently, having been pioneered in the early 1980s, the efficiency of the process is far from ideal. Performing PCR and its variants usually require highly skilled personnel trained to properly execute the technique, a time period spanning several hours to complete the process, and multiple pieces of scientific equipment, such as thermal cyclers, to perform the procedural steps. As a result, although performing PCR and its variants provide accurate results in a period of time considerably faster than that of other diagnostic testing methods, the techniques are difficult, time-consuming, and expensive.

A device is therefore required that enhances the ability for individuals to perform diagnostic testing using the highly effective and accurate methods of PCR and its variants on their own accord and in a less expensive manner. Specifically, due to the RNA-related shortcoming of so-called “traditional” PCR, it is desired that the device be capable of performing RT-PCR so that the number of testable conditions is maximized. It is further desired that the device be small enough so that the average individual would be able to effectively store it when not in use, and be produced in a large-scale capacity such that the device's availability could be extended to areas of the world that are difficult to access, either physically or practically. It is also desired that the device allow for the completion of the diagnostic testing in an expeditious manner, without the assistance of specialized personnel and extensive laboratory equipment.

### SUMMARY OF THE INVENTION

In view of the disadvantages that come with the general laboratory-setting requirement to perform diagnostic testing via PCR and its variants, the present invention is directed to a more readily accessible alternative for individuals to perform the diagnostic testing with ease. The present invention is thus directed to a diagnostic testing device for detecting sequences of genetic material and other targeted molecules by collecting at least one sample, preparing the at least one sample for a reaction, performing the reaction, and indicating the presence or absence of an at least one targeted molecule or sequence of genetic material. Thus, the present invention advantageously provides for a means to perform timely diagnostic testing functions to ensure accurate results without the need for extensive equipment or laboratory infrastructure.

In more specific terms, the diagnostic testing device is comprised of a housing which is further comprised of an exterior component, an interior component, and a testing assembly disposed therein. The exterior component of the device may comprise at least one inlet, at least one viewing window, and an actuation device. In one embodiment, the exterior component may comprise a protective cap, at least one inlet, a first viewing window, a second viewing window, and an actuation device. In another embodiment, the exterior component may comprise a protective cap, at least one inlet, a first viewing window, a second viewing window, a third viewing window, and an actuation device. By way of non-limiting example, the at least one viewing window is disposed to indicate the device's readiness to enter a heating phase. The testing assembly, however, may comprise an entrance component, mixing component, heating component, reaction component, results component, and absorption component. In one embodiment, the components comprising the testing assembly are not separate and distinct from one another. In another embodiment, the components comprising the testing assembly are separate and distinct components.

The exterior component of the device is disposed in fluid communication with the interior component of the device through the at least one inlet, with the at least one inlet configured to accept at least one sample therein. The entrance component within the testing assembly is comprised of a sample collection element, which is structurally configured to receive the at least one sample. By way of non-limiting example, the device may comprise a sample collection element which extends from the entrance component through both the mixing component and the heating component and ends immediately before the reaction component. By way of another non-limiting example, the at least one sample may be a physiological fluid, such as saliva or blood, originating from species including, but not limited to, humans, animals, bacteria, parasites, enveloped and non-enveloped viruses, yeasts, and molds. In at least one embodiment, the at least one sample collected by the sample collection element may volumetrically lie in the range of between generally about 100 to 500 microliters. As used herein, the term “between generally about” refers to the tolerance of values within the standard of error to a person of ordinary skill in the art. The sample collection element is comprised of a filter assembly that may itself comprise at least one fluid channel having a plurality of micropores disposed therein, the filter assembly being configured to convert the at least one sample into a filtered sample as it flows therethrough. By way of non-limiting example, the at least one fluid channel within the sample collection element may comprise paraffin-molded wax channels. The filter assembly acts simultaneously to facilitate the lateral movement of the at least one sample in one direction via hydrophobic-hydrophilic interactions and to remove any non-targeted components of the at least one sample such that the device can convert the at least one sample into the filtered sample as it laterally passes through the entrance component. By way of non-limiting example, the sample collection element may be made of a micro-absorbent paper. In one embodiment, the micro-absorbent paper may comprise specific probes and other nanoparticles with a high affinity to the at least one targeted molecule or sequence of genetic material such that the at least one sample can covalently bond to the specific probes and other nanoparticles.

The mixing component, which is comprised of a mixing pad, is structured such that the mixing pad, which may comprise at least one mixing composition formulated to mix with the filtered sample, will have its contents mix with the filtered sample as the filtered sample interacts with the mixing pad. In doing so, the mixing of the at least one mixing composition with the filtered sample results in a mixed sample that may comprise enriched fragments of genetic material. By way of non-limiting example, the at least one mixing composition may comprise a buffer component, the buffer component comprising at least one buffer, and a reactant component, the reactant component comprising at least one reactant. In one embodiment, the reactant component of the at least one mixing composition comprises magnetic beads such that any targeted nucleic acids and other biological molecules can be recognized and captured by hybridization methods. In one embodiment, the preferred time frame to mix the at least one mixing composition and the filtered sample to form the mixed sample is within the range of generally about one to two minutes. As used herein, the term “generally about” refers to the tolerance of values within the standard of error to a person of ordinary skill in the art.

The heating component, which is comprised of a heating mechanism, is structured such that the heating mechanism applies heat to the interior of the heating component such that the temperature of the mixed sample is increased, producing a heated sample. In one embodiment of the present invention, the heating mechanism is distinct and separable from the heating component. In another embodiment, the heating mechanism is not separable from the heating component. By way of non-limiting example, the heating mechanism may apply convective heat by an exothermic chemical reaction. In one embodiment, the heating mechanism may be activated through utilization of the actuation device. By way of non-limiting example, the actuation device may comprise a depressible button, an electronic control module, or a sensory-sensitive control module. In one embodiment, the heating mechanism is configured to produce temperatures generally between about 60° C. and about 75° C. In one embodiment, the heating mechanism is configured to produce temperatures generally between about 60° C. and about 72° C. In such an embodiment, the preferred temperature to be produced by the heating mechanism is generally about 65° C. In one embodiment, the preferred time frame to produce heat by the heating mechanism is within the range of generally between about fifteen to twenty minutes. As used herein, the term “generally between about” refers to the tolerance of values within the standard of error to a person of ordinary skill in the art.

The reaction component, which is comprised of a reaction matrix, is structured such that the reaction matrix, which may comprise at least one reaction fluid disposed to find the at least one targeted molecule or sequence of genetic material within the heated sample, interacts with the heated sample as it flows therethrough in such a way to produce a reacted sample that allows the device to determine whether the at least one targeted molecule or sequence of genetic material is present or absent. By way of non-limiting example, the at least one reaction fluid can have a varying composition such that a wide variety of targeted sequences of genetic material and other molecules can be discoverable via diagnostic testing with the present invention.

Once the heated sample has sufficiently interacted with the reaction matrix such that any actively reacting components within the reaction component have completed reacting, thus producing the reacted sample, the results of the performed diagnostic testing can be visualized by the results component, which may itself comprise a first indication element and second indication element and which produces a tested sample as it flows therethrough. In one embodiment, the exterior component comprises a second viewing window disposed to indicate completion of an assay and a third viewing window disposed to indicate the presence or absence of the at least one targeted molecule or sequence of genetic material. In such an embodiment, the second viewing window disposed to indicate completion of an assay does so with the first indication element, which indicates completion of the assay via a colorimetric reading. Further, in such an embodiment, the third viewing window disposed to indicate the presence or absence of the at least one targeted molecule or sequence of genetic material does so with the second indication element, which indicates the presence or absence of the at least one targeted molecule or sequence of genetic material via formation of a signal, usually in the form of a line.

Once the results of the performed diagnostic testing are visualized in the at least one viewing window of the exterior component of the device, the tested sample interacts with the absorption component, which may itself comprise an absorbent pad. In doing so, the tested sample is absorbed by the absorbent pad in order to collect any of the remaining tested sample and to halt the movement of the tested sample as it flows therethrough. This device thus allows for the detection and diagnosis of a variety of conditions in a mobile housing that is laboratory-independent. As should be apparent, the device's ability to efficiently and cost-effectively function as a diagnostic test is extremely beneficial to the field of medicine and many other potential outlets.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a schematic, top perspective view of one embodiment of the reverse transcription polymerase chain reaction diagnostic testing device at a slightly downward angle.

FIG. 2 is a schematic, top perspective view of one embodiment of the testing assembly disposed within the housing of the reverse transcription polymerase chain reaction diagnostic testing device at a slightly downward angle.

FIG. 3 is a schematic, top perspective view of one embodiment of an attachable member to the testing assembly.

FIG. 4 is a flow diagram of the various samples identified at different stages within the present invention.

FIG. 5 is a schematic, top perspective view of another embodiment of the testing assembly disposed within the housing of the polymerase chain reaction diagnostic testing device.

FIG. 6 is a schematic, top perspective view of another embodiment of the reverse transcription polymerase chain reaction diagnostic testing device at a slightly downward angle.

Like reference numerals refer to like parts throughout the several views of the drawings.

### DETAILED DESCRIPTION OF THE PREFERRED EMBODIMENT

The invention now will be described more fully hereinafter with reference to the accompanying drawings in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

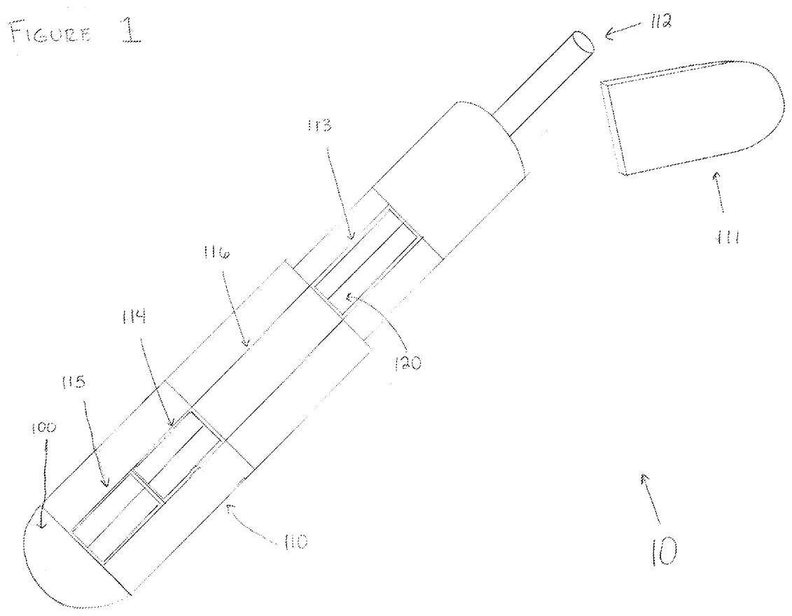

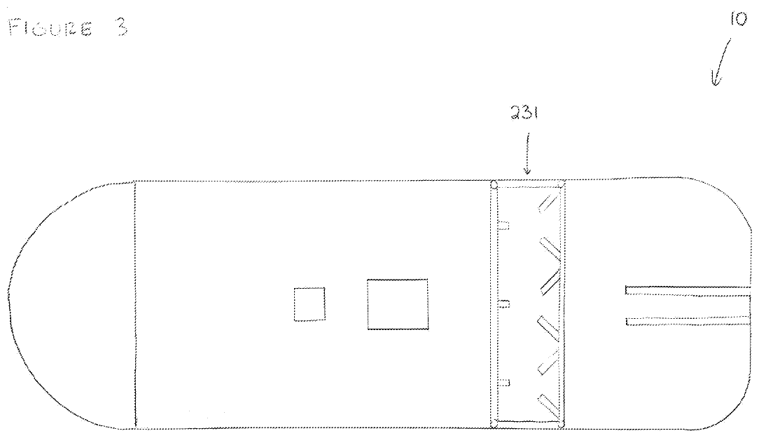

Turning now descriptively to the figures, FIGS. 1, 2, and 3 illustrate an inventive diagnostic testing device configured to detect and diagnose both infectious and non-infectious conditions through utilization of reverse transcription polymerase chain reaction (RT-PCR).

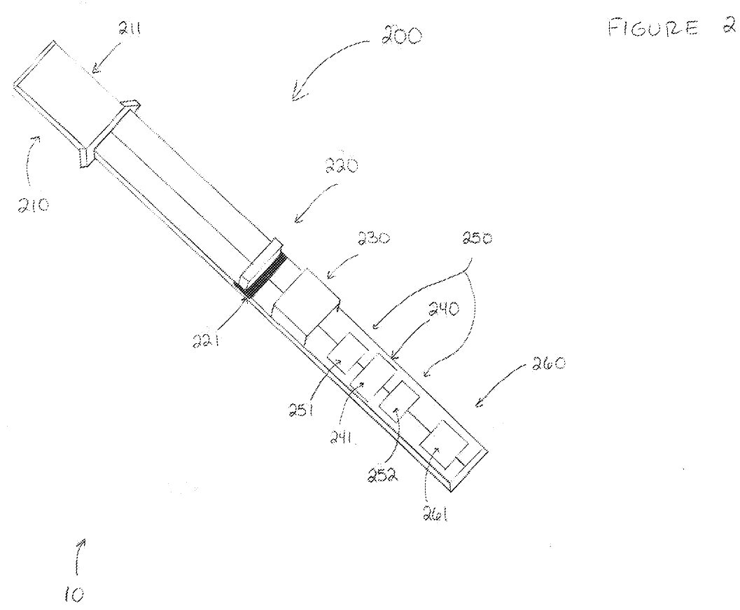

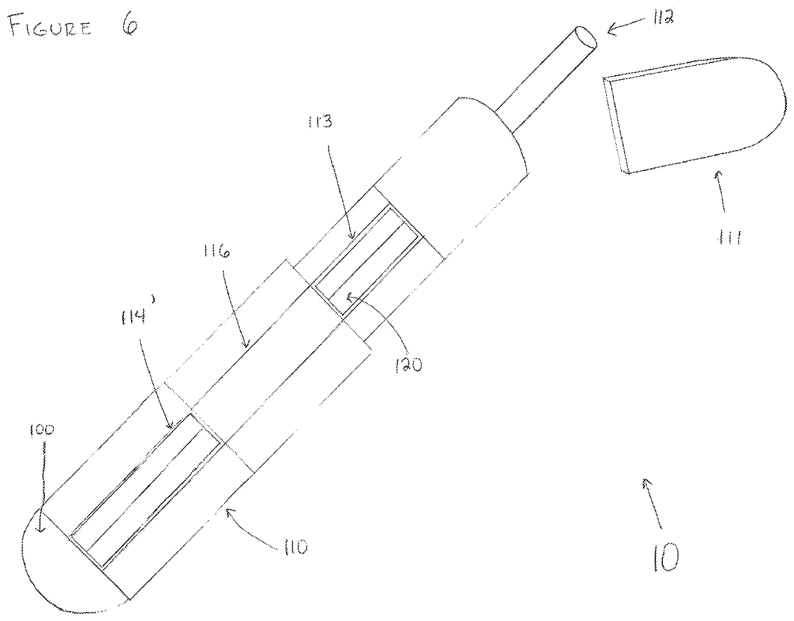

FIGS. 1, 2, and 3 show that the RT-PCR diagnostic testing device 10 is primarily comprised of a housing 100 which is further comprised of an exterior component 110, an interior component 120, and a testing assembly 200 disposed therein. The exterior component 110 may comprise at least one inlet 112, a first viewing window 113, a second viewing window 114, a third viewing window 115, and an actuation device 116. In one embodiment of the present invention, illustrated in FIG. 6, the exterior component 110 may comprise a protective cap 111, at least one inlet 112, a first viewing window 113, a second viewing window 114′, and an actuation device 116. The testing assembly 200, illustrated in FIG. 2, however, may comprise an entrance component 210, a mixing component 220, a heating component 230, a reaction component 240, a results component 250, and an absorption component 260. In one embodiment of the present invention, illustrated in FIG. 2, the plurality of components comprising the testing assembly 200 are not separate and distinct from one another.

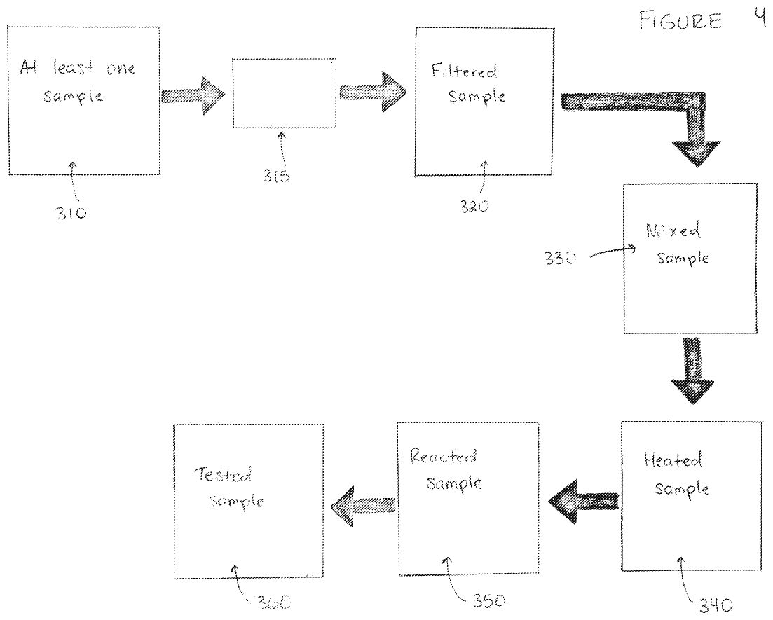

As disclosed in FIG. 1, the exterior component 110 is disposed in fluid communication with the interior component 120 through the at least one inlet 112, with the at least one inlet 112 configured to accept at least one sample 310 therein, as will be explained in greater detail with reference to FIG. 4. With reference to FIGS. 2 and 4, the entrance component 210 within the testing assembly 200 is comprised of a sample collection element 211, which is structurally configured to receive the at least one sample 310. In one embodiment of the present invention, the device 10 may comprise a sample collection element 211 which extends from the entrance component 210 through both the mixing component 220 and the heating component 230, ending immediately before the reaction component 240. By way of non-limiting example, the at least one sample 310 may be a physiological fluid, such as saliva or blood, originating from species including, but not limited to, humans, animals, bacteria, parasites, enveloped and non-enveloped viruses, yeasts, and molds. In at least one embodiment of the present invention, the at least one sample 310 collected by the sample collection element 211 may volumetrically lie in the range of between generally about 100 to 500 microliters. As used herein, the term “between generally about” refers to the tolerance of values within the standard of error to a person of ordinary skill in the art. As disclosed in FIGS. 2 and 4, the sample collection element 211 is comprised of a filter assembly 315 that may itself comprise at least one fluid channel having a plurality of micropores disposed therein, the filter assembly 315 being configured to convert the at least one sample 310 into a filtered sample 320 as it flows therethrough. By way of non-limiting example, the at least one fluid channel within the sample collection element 211 may comprise paraffin-molded wax channels. With reference to FIGS. 2 and 4, the filter assembly 315 acts simultaneously to facilitate the lateral movement of the at least one sample 310 in one direction via hydrophobic-hydrophilic interactions and to remove any non-targeted components of the at least one sample 310 such that the device 10 can convert the at least one sample 310 into the filtered sample 320 as it passes through the entrance component 210. By way of non-limiting example, the sample collection element 211 may be made of a micro-absorbent paper. In one embodiment, the micro-absorbent paper may comprise specific probes and other nanoparticles with a high affinity to the at least one targeted molecule or sequence of genetic material such that the at least one sample 310 can covalently bond to the specific probes and other nanoparticles.

With primary reference to FIGS. 2, 4, and 5, the mixing component 220, which is comprised of a mixing pad 221, is structured such that the mixing pad 221, which may comprise at least one mixing composition formulated to mix with the filtered sample 320, will have its contents mix with the filtered sample 320 as the filtered sample 320 interacts with the mixing pad 221. In doing so, the mixing of the at least one mixing composition with the filtered sample 320 results in a mixed sample 330 that may comprise enriched fragments of genetic material. In one embodiment of the present invention, upon assembly of the device 10, the mixing pad 221 may comprise the at least one mixing composition. In another embodiment, the mixing pad 221 may not comprise the at least one mixing composition upon assembly of the device 10. By way of non-limiting example, the at least one mixing composition may comprise a buffer component, the buffer component comprising at least one buffer, and a reactant component, the reactant component comprising at least one reactant. In one embodiment, the reactant component of the at least one mixing composition comprises magnetic beads such that any targeted nucleic acids and other biological molecules can be recognized and captured by hybridization methods. In one embodiment, the preferred time frame to mix the at least one mixing composition and the filtered sample 320 to form the mixed sample 330 is within the range of generally about one to two minutes. As used herein, the term “generally about” refers to the tolerance of values within the standard of error to a person of ordinary skill in the art. In one embodiment of the present invention, upon completion of the mixing of the at least one mixing composition with the filtered sample 320 to result in the mixed sample 330, the first viewing window 113 of the device 10 may be disposed to indicate both completion of the mixing of the at least one mixing composition with the filtered sample 320 and also the readiness of the device 10 to enter a heating phase. In such an embodiment, illustrated in FIGS. 1 and 6, the first viewing window 113 may indicate both completion of the mixing of the at least one mixing composition with the filtered sample 320 and also the readiness of the device 10 to enter a heating phase via a colorimetric reading.

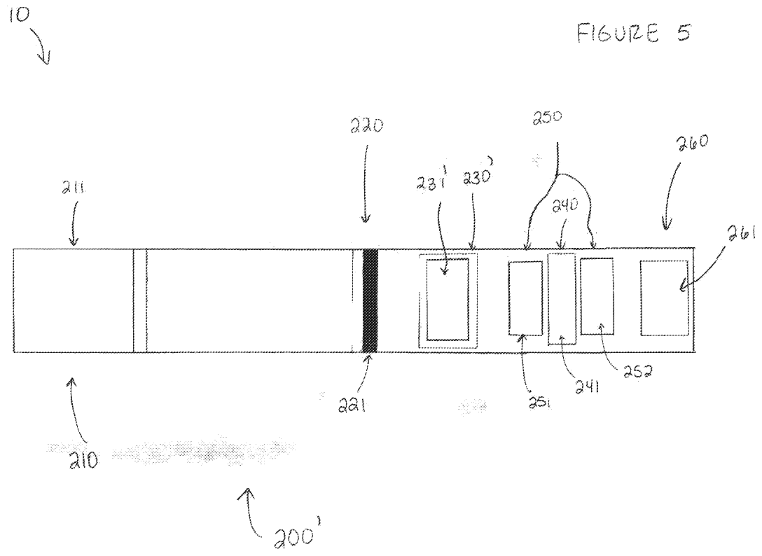

The heating component 230, which is comprised of a heating mechanism 231, is structured such that the heating mechanism 231 applies heat to the interior of the heating component 230 such that the temperature of the mixed sample 330 is increased, producing a heated sample 340, as represented in FIG. 4. In one embodiment of the present invention, illustrated in FIG. 2, the heating mechanism 231 is distinct and separable from the heating component 230. In another embodiment, illustrated in FIG. 5, the heating mechanism 231′ within the testing assembly 200′ is not separable from the heating component 230′. By way of non-limiting example, the heating mechanism 231 may apply convective heat by an exothermic chemical reaction. In one embodiment, the heating mechanism 231 may be activated through utilization of the actuation device 116. By way of non-limiting example, the actuation device 116 may comprise a depressible button, an electronic control module, or a sensory-sensitive control module. In one embodiment, the heating mechanism 231 is configured to produce temperatures generally between about 60° C. and about 75° C. In another embodiment, the heating mechanism 231 is configured to produce temperatures generally between about 60° C. and about 72° C. In such an embodiment, the preferred temperature to be produced by the heating mechanism 231 is generally about 65° C. In one embodiment, the preferred time frame to produce heat by the heating mechanism 231 is within the range of generally between about fifteen to twenty minutes.

With primary reference to FIGS. 2, 4, and 5, the reaction component 240, which is comprised of a reaction matrix 241, is structured such that the reaction matrix 241, which may comprise at least one reaction fluid disposed to find the at least one targeted molecule or sequence of genetic material within the heated sample 340, interacts with the heated sample 340 as it flows therethrough in such a way to produce a reacted sample 350 that allows the device 10 to determine whether the at least one targeted molecule or sequence of genetic material is present or absent. In one embodiment of the present invention, the presence of absence of the at least one targeted molecule or sequence of genetic material is visually determined by staining the reacted sample 340 with a chemical dye. By way of non-limiting example, the at least one reaction fluid can have a varying composition such that a wide variety of targeted sequences of genetic material and other molecules can be discoverable via diagnostic testing with the present invention.

As disclosed in FIGS. 2, 4, and 5, once the heated sample 340 has sufficiently interacted with the reaction matrix 241 such that any actively reacting components within the reaction component 240 have completed reacting, thus producing the reacted sample 350, the results of the performed diagnostic testing can be visualized by the results component 250, which may itself comprise a first indication element 251 and second indication element 252 and which produces a tested sample 360 as it flows therethrough. In one embodiment, the exterior component 110 comprises a second viewing window 114 disposed to indicate completion of an assay and a third viewing window 115 disposed to indicate the presence or absence of the at least one targeted molecule or sequence of genetic material. In such an embodiment, the second viewing window 114 disposed to indicate completion of an assay does so with the first indication element 251, which indicates completion of the assay via a colorimetric reading. Further, in such an embodiment, the third viewing window 115 disposed to indicate the presence or absence of the at least one targeted molecule or sequence of genetic material does so with the second indication element 252, which indicates the presence or absence of the at least one targeted molecule or sequence of genetic material via formation of a signal, usually in the form of a line. In another embodiment, illustrated in FIG. 6, the exterior component 110 comprises a second viewing window 114′ disposed to both indicate completion of an assay via a colorimetric reading with the first indication element 251 and to indicate the presence or absence of the at least one targeted molecule or sequence of genetic material via formation of a signal, usually in the form of a line, with the second indication element 252.

Once any results are visualized in the at least one viewing window of the exterior component 110 of the device 10, the tested sample 360 continues to laterally flow in the same direction and interacts with the absorption component 260, which may itself comprise an absorbent pad 261. In doing so, the tested sample 360 is absorbed by the absorbent pad 261 in order to collect any of the remaining tested sample 360 and other potential fluids, and to halt the movement of the tested sample 360 as it flows therethrough.

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A diagnostic testing device for detecting sequences of genetic material comprising:
a housing comprising an exterior component disposed in fluid communication with an interior component through at least one inlet, said at least one inlet configured to accept at least one sample therein; and
said housing further comprising a testing assembly disposed therein, said testing assembly comprising a plurality of components serially disposed in fluid communication for the flow of a sample therethrough, said plurality of components comprising:
an entrance component comprising a sample collection element structurally configured to receive at least one sample,
a mixing component comprising a mixing pad, said mixing pad comprising at least one mixing composition formulated to mix with a filtered sample,
a reaction component comprising a reaction matrix, and
a results component comprising at least one indication element.

2. The device of claim 1, wherein said sample collection element comprises a filter assembly configured to convert the sample into the filtered sample as it flows therethrough.

3. The device of claim 2, wherein said filter assembly comprises at least one fluid channel having a plurality of micropores disposed therein.

4. The device of claim 1, wherein said at least one mixing composition comprises a buffer component, said buffer component comprising at least one buffer, and a reactant component, said reactant component comprising at least one reactant.

5. The device of claim 1, wherein said exterior component comprises at least one viewing window, said at least one viewing window configured to indicate detection of sequences of genetic material.

6. A diagnostic testing device for detecting sequences of genetic material comprising:
a housing comprising an exterior component disposed in fluid communication with an interior component through at least one inlet, said at least one inlet configured to accept at least one sample therein; and
said housing further comprising a testing assembly disposed therein, said testing assembly comprising a plurality of components serially disposed in fluid communication for the flow of a sample therethrough, said plurality of components comprising:
an entrance component comprising a sample collection element structurally configured to receive at least one sample,
a heating component comprising a heating mechanism,
a reaction component comprising a reaction matrix, and
a results component comprising at least one indication element.

7. The device of claim 6, wherein said sample collection element comprises a filter assembly configured to convert the sample into a filtered sample as it flows therethrough.

8. The device of claim 7, wherein said filter assembly comprises at least one fluid channel having a plurality of micropores disposed therein.

9. The device of claim 6, wherein said heating mechanism applies convective heat to the interior of said heating component by an exothermic chemical reaction.

10. The device of claim 6, wherein said heating mechanism is configured to produce temperatures generally between about 60° C. and about 75° C.

11. The device of claim 6, wherein said heating mechanism is configured to produce temperatures generally between about 60° C. and about 65° C.

12. The device of claim 6, wherein said exterior component comprises at least one viewing window, said at least one viewing window configured to indicate detection of sequences of genetic material.

13. The device of claim 6, wherein said exterior component comprises an actuation device.

14. A diagnostic testing device for detecting sequences of genetic material comprising:
a housing comprising an exterior component disposed in fluid communication with an interior component through at least one inlet, said at least one inlet configured to accept at least one sample therein; and
said housing further comprising a testing assembly disposed therein, said testing assembly comprising a plurality of components serially disposed in fluid communication for the flow of a sample therethrough, said plurality of components comprising:
an entrance component comprising a sample collection element structurally configured to receive at least one sample,
a mixing component comprising a mixing pad, said mixing pad comprising at least one mixing composition formulated to mix with a filtered sample,
a heating component comprising a heating mechanism,
a reaction component comprising a reaction matrix,
and a results component comprising at least one indication element.

15. The device of claim 14, wherein said sample collection element comprises a filter assembly configured to convert the sample into the filtered sample as it flows therethrough.

16. The device of claim 15, wherein said filter assembly comprises at least one fluid channel having a plurality of micropores disposed therein.

17. The device of claim 14, wherein said at least one mixing composition comprises a buffer component, said buffer component comprising at least one buffer, and a reactant component, said reactant component comprising at least one reactant.

18. The device of claim 14, wherein said heating mechanism applies convective heat to the interior of said heating component by an exothermic chemical reaction.

19. The device of claim 14, wherein said heating mechanism is configured to produce temperatures generally between about 60° C. and about 75° C.

20. The device of claim 14, wherein said heating mechanism is configured to produce temperatures generally between about 60° C. and about 65° C.

21. The device of claim 14, wherein said exterior component comprises at least one viewing window, said at least one viewing window configured to indicate detection of sequences of genetic material.

22. The device of claim 14, wherein said exterior component comprises an actuation device.
